(19) United States
(12) Patent Application Publication
(10) Pub. No.: US 2011/0034765 A1
(43) Pub. Date: Feb. 10, 2011

(54) Endoscopic Instrument

(75) Inventor: Frank WEHRHEIM, Bretten (DE)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: RICHARD WOLF GMBH, Knittlingen (DE)

(21) Appl. No.: 12/849,150

(22) Filed: Aug. 3, 2010

(30) Foreign Application Priority Data
Aug. 6, 2009 (DE) 10 2009 036 424.2

(51) Int. Cl. A61B 1/00; B29C 63/06

(52) U.S. Cl. 600/104; 264/104

(57) ABSTRACT

An endoscopic instrument has a shaft designed in a flexible manner along its longitudinal axis in at least one section. The flexible section of the shaft has a tube-shaped wall made of an electroactive polymer with a plurality of control electrodes and reference electrodes respectively embedded therein. As seen in the axial direction, the control electrodes and reference electrodes are respectively arranged alternately and separated from one another. The control electrodes and reference electrodes alternating in the axial direction respectively have a rigid design and are respectively interconnected in an electrically conducting manner in the axial direction by elastic webs arranged on the outer or inner circumference. A method is also provided for producing a flexible section of such an endoscopic instrument.

### BACKGROUND OF THE INVENTION

The invention relates to an endoscopic instrument having a shaft designed in a flexible manner along its longitudinal axis (X) in at least one section, wherein the flexible section of the shaft has a tube-shaped wall made of an electroactive polymer with a plurality of control electrodes and reference electrodes embedded therein and respectively arranged alternately as seen in the axial direction (X) and separated from one another.

By way of example, German published patent application DE 10 2008 047 776 A1 discloses an endoscopic instrument having a shaft section made of an electroactive polymer, wherein electrodes for activation are embedded into the electroactive polymer. These electrodes are designed as structures engaging into one another in a tooth-like manner.

### BRIEF SUMMARY OF THE INVENTION

It is the object of the present invention to improve such an endoscopic instrument, such that improved flexibility is attained in the case of an electrode structure that can be designed in a simple manner.

This object is achieved by an endoscopic instrument of the type described at the outset, wherein the control electrodes and reference electrodes alternating in the axial direction (X) respectively have a rigid design and are respectively interconnected in an electrically conducting manner in the axial direction (X) by elastic webs arranged on the outer or inner circumference. Preferred embodiments are described in the subsequent description and the drawings.

The endoscopic instrument according to an embodiment of the invention has a shaft, which is designed in a flexible manner in at least one axial section, i.e., in a section along the longitudinal axis thereof. This flexible section has a tube-shaped wall made of an electroactive polymer. Embedded into the electroactive polymer is a plurality of reference electrodes, by which a voltage can be applied to the electroactive polymer in order to cause the electroactive polymer to change its shape and thus bend the shaft. For this purpose, the control electrodes and reference electrodes are arranged alternately in the axial direction, i.e., one control electrode is respectively situated between two reference electrodes. At the same time, the electrodes are spaced apart, such that situated therebetween is the electroactive polymer, which changes its shape due to the application of an electronic voltage.

According to an embodiment of the invention, the control electrodes and reference electrodes respectively have a rigid design and are respectively interconnected in an electrically conducting manner in the axial direction by elastic webs, i.e., webs that can be bent without lasting deformation. Here, the control electrodes are interconnected among themselves by elastic webs, and the reference electrodes are interconnected among themselves in an electrically conducting manner by elastic webs. There is no electrical connection between the control electrodes and reference electrodes. The elastic webs allow relative motion in the axial direction between the rigid control electrodes and reference electrodes in the case of compression or expansion of the electroactive polymer situated therebetween.

The rigid design of the control electrodes and reference electrodes achieves stabilization of the electroactive polymer wall in the radial direction in relation to the longitudinal axis of the shaft. The electrical connection of the plurality of reference electrodes among themselves and the electrical connection of the control electrodes among themselves moreover reduces the number of required connection lines, as a result of which a simplified design of the electrode structure is produced.

Moreover, according to an embodiment of the invention, the elastic webs are situated either on the outer circumference or on the inner circumference of the reference electrodes or control electrodes. That is to say, the elastic webs are in respect of the longitudinal axis of the shaft arranged lying radially inside of the electrodes or radially outside on the outer sides of the electrodes. This design ensures good flexibility of the shaft. The reference electrodes preferably all extend parallel to one another. Accordingly, the control electrodes preferably also extend parallel to one another, more particularly the control electrodes and the reference electrodes also extend parallel to one another. This produces parallel layering of the individual electrodes in the wall made of electroactive polymer.

The control electrodes and/or the reference electrodes preferably have a plate-shaped design and extend transversely with respect to the longitudinal axis of the shaft. Hence, the surfaces of the plate-shaped electrodes extend in diameter planes in relation to the longitudinal axis of the shaft. The plate-shaped structure results from the fact that the electrodes have a greater extent in the radial direction than in the axial direction, i.e., in the direction parallel to the longitudinal axis. The electrodes in any case preferably have a larger extent in the circumferential direction, i.e., they extend over a large circumferential area, possibly over the entire circumference of the wall. The plate-shaped embodiment produces a large active electrode surface, and at the same time greater stability of the wall is obtained in the radial direction as a result of the greater electrode thickness in the radial direction.

Furthermore, as seen in the circumferential direction, the flexible section is expediently divided into at least two actuator fields that can be activated separately. In these fields, as seen in the axial direction in each case, the control electrodes and reference electrodes described above are arranged in an alternating manner and at a distance from one another. More preferably, provision is made for three or more actuator fields, which are preferably arranged in a uniformly distributed manner in the circumferential direction in the wall. As a result of it being possible for the actuator fields to be activated separately, i.e., for the electrodes thereof to be actuated separately with voltage, it is then possible for the shaft to be bent in any direction, depending on which actuator field(s) is(are) activated. As described above, the electrodes are thus arranged in an alternating arrangement in the actuator fields of the control electrodes and reference electrodes, with the electrical connections by elastic webs. The individual actuator fields are each preferably designed such that the electrodes of the individual actuator fields together extend preferably substantially over the entire circumference of the wall. This achieves the largest possible stabilization of the wall in the radial direction by the electrodes.

The reference electrodes of the plurality of actuator fields are preferably interconnected in an electrically conducting manner. This can reduce the number of connection lines, because the reference electrodes of a plurality of, preferably of all, actuator fields can thus be electrically connected over a common connection line.

For this, the reference electrodes of the plurality of actuator fields respectively situated in the same diameter plane relative to the longitudinal axis of the shaft are, more preferably, interconnected in the circumferential direction in an electrically conducting manner. The reference electrodes situated in different diameter planes are connected in the axial direction by the above-described elastic webs.

More preferably, the reference electrodes of the plurality of actuator fields respectively situated in one diameter plane are also interconnected in the circumferential direction in a mechanical manner. The connection expediently produces both the mechanical and electrical connection. A mechanical stabilization of the shaft wall is thus obtained at the same time by the electrical connection in the circumferential direction, because the reference electrodes form annular arrangements in this manner. However, compared to the actual electrode surfaces of the reference electrodes, the connections between the individual actuator fields, if need be, manufactured in a thinned manner in the radial direction, such that a certain amount of flexibility is obtained in the circumferential direction, which flexibility, as described below, can be advantageous for the production in particular.

The reference electrodes of the plurality of actuator fields more preferably have a common electrical connection in the axial direction. That is to say, when the reference electrodes situated in the same diameter plane are interconnected in an electrically conducting manner in the circumferential direction, it suffices for two reference electrodes, which are adjacent to one another in the axial direction and have a control electrode situated therebetween, to be interconnected in the axial direction in only one of the actuator fields by an elastic web. Then, such elastic webs for connecting these two reference electrodes or reference electrode rings are not required in the other actuator fields. This reduces the number of axial connections between the reference electrodes. On the one hand, this increases the flexibility of the instrument and, on the other hand, this simplifies the production of the electrode structure.

Furthermore, it is preferable for two reference electrodes adjacent to one another in the axial direction to be interconnected by an elastic web, which, as seen in the circumferential direction, is connected to the first end of a first reference electrode and an opposite second end of the second adjacent reference electrode. Here, as seen in a radial projection, the web crosses the control electrode situated between the reference electrodes, but extends laterally past the inner or outer circumference of the control electrode without contacting the latter. The elastic web and the connected reference electrodes form a Z-shaped structure. This structure ensures the elasticity of the web and the axial mobility of the two reference electrodes with respect to one another.

The common axial connections of the individual reference electrodes are more preferably arranged alternately distributed in the plurality of actuator fields. That is to say that, for the multiplicity of reference electrodes situated above one another in the axial direction, the axial connections between the individual reference electrodes are not all arranged in a single actuator field, but are distributed over a plurality of, preferably over all, actuator fields. By way of example, this allows the formation of an overall helical profile over the circumference of the wall for the axial connection between the reference electrodes. This ensures uniform flexibility of the shaft in all radial directions. There is no bending direction that would be affected more than the others by the arrangement of the electrical connection between the reference electrodes.

Hence, it is particularly preferable for the axial connection of the reference electrodes to be designed as a web running over the wall or embedded therein in a helical manner. This web runs either on the inner circumference of the reference electrodes or on the outer circumference of the reference electrodes and interconnects all reference electrode rings in the axial direction.

Further preferably, in each case, two control electrodes adjacent to one another in an individual actuator field are also interconnected by an elastic web, which, as seen in the circumferential direction, is connected to the first end of a first control electrode and an opposite second end of the second adjacent control electrode. Then, a reference electrode is situated respectively between the two adjacent control electrodes, with the web connecting the control electrodes crossing the reference electrodes, as seen in the radial projection, but passing the reference electrode laterally at the outer or inner circumference. The connection described in this manner between two control electrodes likewise provides a Z-shaped structure consisting of two control electrodes and the web situated therebetween.

When all control electrodes, situated above one another in the axial direction, of an actuator field are connected in this manner, it thus provides a zigzag-shaped electrode structure overall, with the legs of the zigzag-shaped structure situated horizontally in the case of a vertical alignment of the longitudinal axis being formed by the control electrodes and the legs running at an angle being formed by the elastic webs. This likewise obtains good elasticity and deformability of the electrical connection webs, such that the control electrodes can move toward one another or can move apart when there is deformation of the electroactive polymer.

It is particularly preferable for the elastic webs, which interconnect the reference electrodes, and/or the elastic webs, which interconnect the control electrodes, to be arranged on the circumferential side of the control electrodes and reference electrodes situated radially on the inside. More preferably, both the webs interconnecting the control electrodes and the webs interconnecting the reference electrodes are situated on the radially inward, i.e., inner, circumferential side of the control electrodes and reference electrodes, or of the wall. This protects the electrodes in the interior, and the deformation paths, which the elastic webs have to perform during bending, are minimized.

The invention moreover relates to a method for producing an endoscopic instrument as per the preceding description. According to this method, the section of the shaft designed to be flexible is manufactured such that the arrangement of the control electrodes and reference electrodes, and also the elastic webs connecting these, is first designed as a structure in a flat plane. Here, the control electrodes and reference electrodes alternate in the direction of extent of the plane, i.e., in the case where the electrodes, as described above, are designed in a plate-like shape, the surfaces of the plates extend perpendicular to the plane in which the structure of the control electrodes and reference electrodes is formed.

The structure is subsequently molded into an electroactive polymer, such that an even plate made of electroactive polymer with embedded electrodes is provided. In the next step, this structure consisting of electroactive polymer and molded electrodes is bent in a tube-shaped manner to form a tube-shaped wall. This allows a relatively simple production of the tube-shaped configuration with the embedded electrodes. Then, the tube-shaped wall is preferably encapsulated by an elastic polymer on its outer side after the bending. This then holds together the bent wall on the outside, as described above.

So that it is possible to be able to bend the electrode structure formed in the plane in a ring or tube-shaped manner, it is preferable for the above-described electrical connections between the reference electrodes of the individual actuator fields, which run in the circumferential direction, to be designed to be thinner in the radial direction than the reference electrodes, such that there is bending in these regions, and the reference electrodes and control electrodes themselves do not have to be bent.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is an overall, perspective view of an endoscopic instrument according to an embodiment of the invention;

FIG. 2 is a schematic representation showing the arrangement of electrodes in a flexible section of the instrument according to FIG. 1;

FIG. 3 is a perspective view showing the arrangement of control electrodes and reference electrodes according to an embodiment of the invention;

FIG. 4 is an enlarged section from the embodiment of FIG. 3;

FIGS. 5a-5c are perspective schematic representations showing the deformation of the shaft according to an embodiment of the invention; and

FIG. 6 is an overall perspective view showing an arrangement of the electrodes in the shaft according to an embodiment of the invention.

### DETAILED DESCRIPTION OF THE INVENTION

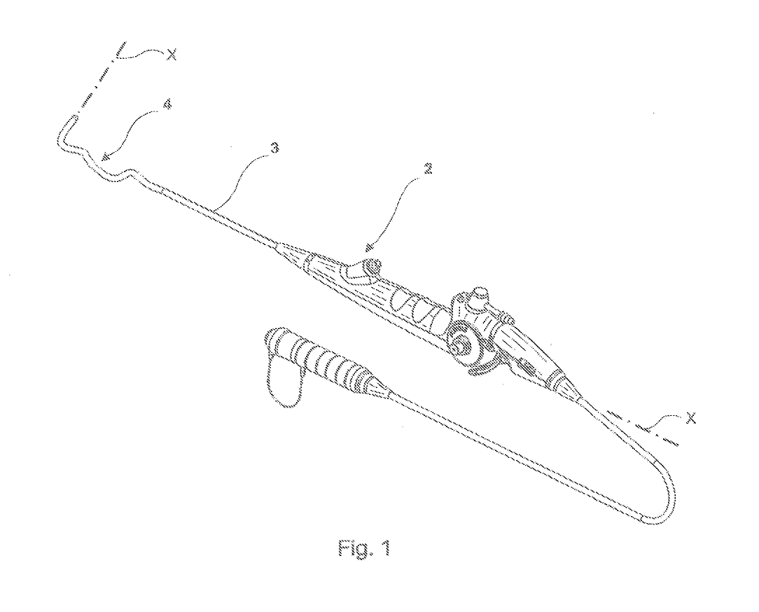

FIG. 1 shows an example of an endoscopic instrument according to the invention. Here, this is an endoscope 2 with a shaft 3, the distal section 4 of which is designed to be flexible or able to bend. According to the invention, the distal section 4 is formed from a tube-shaped wall made of an electroactive polymer, with control electrodes 6 and reference electrodes 8 embedded therein. An electrical voltage can be applied to the electroactive polymer by the control electrodes 6 and reference electrodes 8, as a result of which a deformation of the electroactive polymer is obtained. This deformation is used for the deflection or bending of the distal section 4 of the endoscope 2.

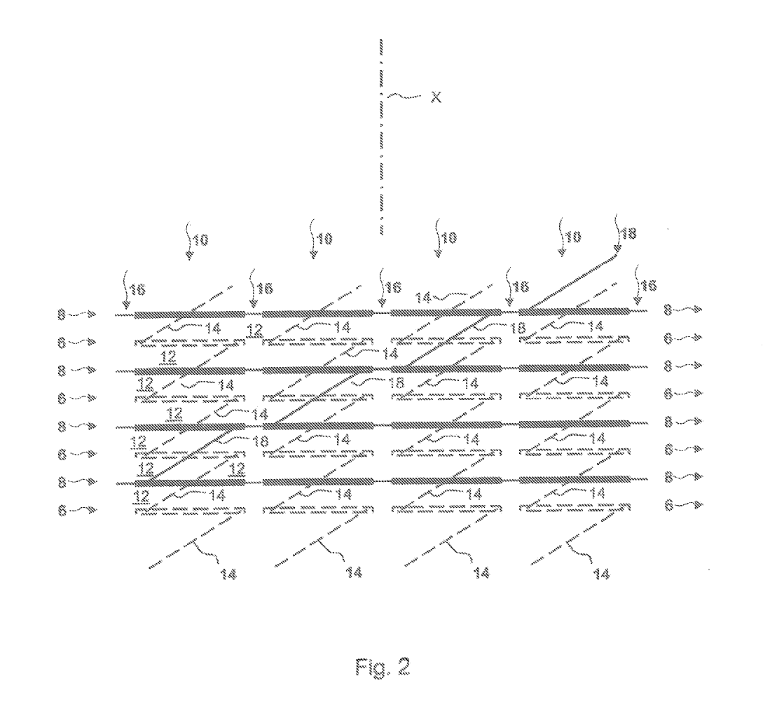

FIG. 2 schematically shows the arrangement, according to an embodiment of the invention, of reference electrodes 8 and control electrodes 6. Distributed about the longitudinal axis X of the shaft 3, a plurality of actuator fields 10 are arranged in the shaft wall of the deformable or flexible section 4, which actuator fields 10 form filaments that can contract. Arranged alternately above one another in the axial direction in the individual actuator fields are in each case the control electrodes 6 and the reference electrodes 8, wherein the reference electrodes 8 and the control electrodes 6 are respectively spaced apart from each other, such that the electroactive polymer can be arranged in the interspaces 12 between the electrodes thus formed. For this, the electrodes 6 and 8 are embedded into the electroactive polymer.

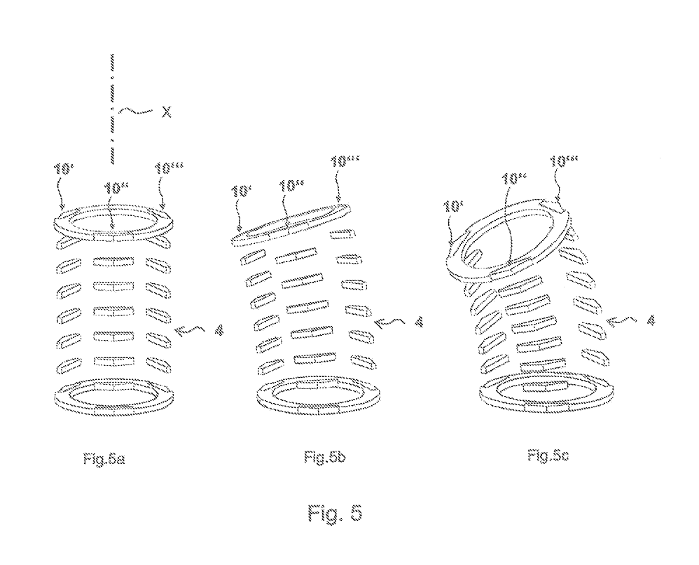

The electroactive polymer in the interspaces 12 is made to contract by applying a voltage between the control electrodes 6 and the reference electrodes 8, such that the entire actuator field 10 or the entire filament thus formed is contracted, and the flexible section 4 is deflected in the corresponding direction, as shown on the basis of FIGS. 5a to 5c. An example with three filaments is illustrated schematically therein. In FIG. 5a, none of the filaments 10′, 10″ or 10′″ is contracted, i.e., no voltage has been applied to the corresponding control electrodes 6 and reference electrodes 8. In the illustration as per FIG. 5b, the filament 10′ has contracted by the application of a voltage on the electrodes thereof, and so the shaft is deflected toward the filament 10′. In the example according to FIG. 5c, the filaments 10′ and 10″ have contracted by applying voltage to their electrodes, and so the shaft or the flexible section 4 thereof is deflected in an angular direction between the filaments 10′ and 10″.

According to an embodiment of the invention, the control electrodes 6 of each of the actuator fields 10 are in each case interconnected in an electrically conducting manner via elastic webs 14, as shown in FIG. 2. The elastic webs 14 extend at an angle between two control electrodes 6, which are adjacent to one another and parallel to one another, such that, as seen in a radial projection on the longitudinal axis X, two adjacent control electrodes 6 and the web 14 situated therebetween form a Z-shaped structure. The multiplicity of control electrodes 6 layered above one another with the elastic webs 14 situated therebetween thus forms a zigzag-shaped structure. This electrical connection of all control electrodes 6 of an actuator field 10 affords the possibility of applying voltage simultaneously to all control electrodes 6 using a single connection line. As a result of the control electrodes 6 of the individual actuator fields 10 being in each case separate from one another, voltage can be applied separately to the control electrodes 6 of the individual actuator fields 10 and hence each actuator field 10 can be activated separately.

In the shown example, only a plurality of actuator fields 10 are arranged in a circumferential manner about the longitudinal axis X in the wall of the flexible section 4. However, it is to be understood that a plurality of actuator fields 10 that can be activated separately can also be arranged one behind the other in the axial direction X, in order to obtain snakelike bending of the distal section 4, as shown in FIG. 1.

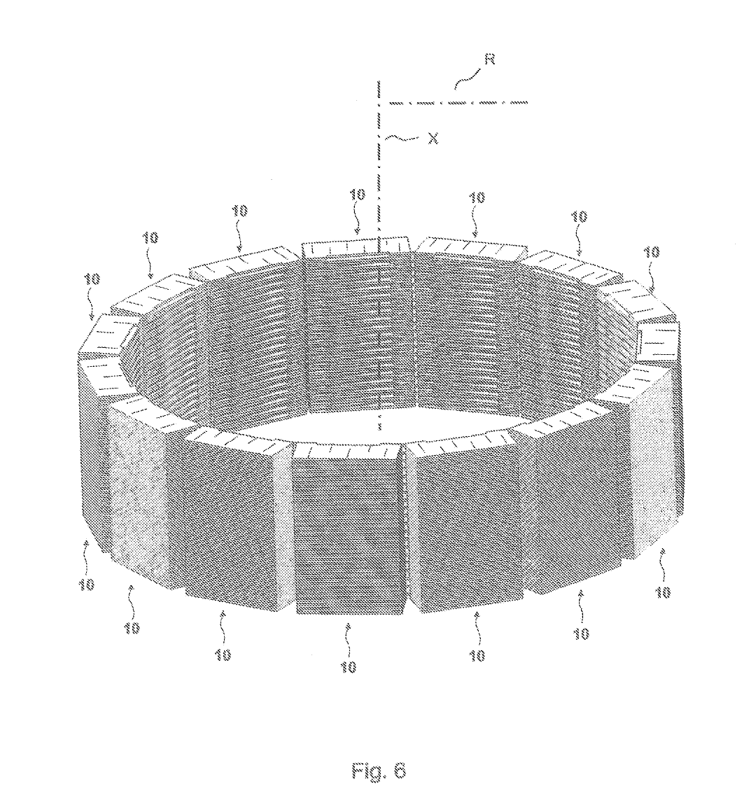

FIG. 2 moreover shows that the reference electrodes 8, which in each case are situated in the same diameter plane relative to the longitudinal axis X, are interconnected in the circumferential direction via webs 16, such that the reference electrodes 8 of a plurality of, preferably of all, actuator fields 10 are interconnected. This provides ring-shaped structures of reference electrodes 8 situated one above the other with webs 16 situated therebetween. Compared to the reference electrodes 8, the webs 16 have a thinner design, such that bending is possible in this region in order to bend the electrode arrangement into the tube-shaped form shown in FIG. 6.

Elastic webs 18 are provided for an electrically conducting interconnection between the reference electrodes 8 or the rings formed from reference electrodes 8 situated one above the other in the axial direction. Like the elastic webs 14, the elastic webs 18 extend at an angle between two adjacent reference electrodes 8, such that a first reference electrode 8 is contacted by the web 18 at a first circumferential end, and the second adjacent reference electrode 8 is contacted by the elastic web 18 at the opposite circumferential end. Since all reference electrodes 8 are interconnected over a plurality of actuator fields 10, it is not necessary to interconnect all reference electrodes 8 in each of the actuator fields 10 by elastic webs 18. Rather, two mutually adjacent ring-shaped arrangements of reference electrodes 8 are only interconnected via an elastic web 18 in one of the actuator fields 10. At the same time, these elastic webs 18 are distributed over the individual actuator fields 10, such that they are always offset by one actuator field 10, and so overall a substantially helical profile around the electrode arrangement is formed by the elastic webs 18.

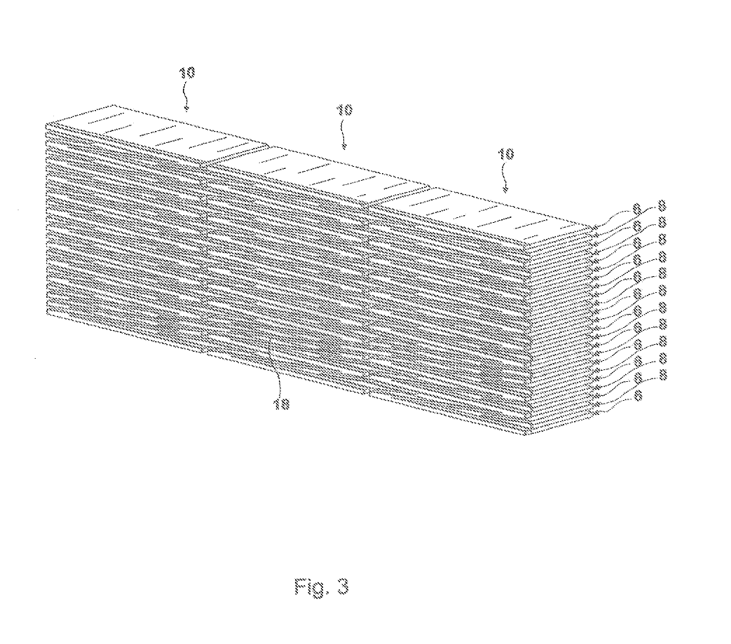

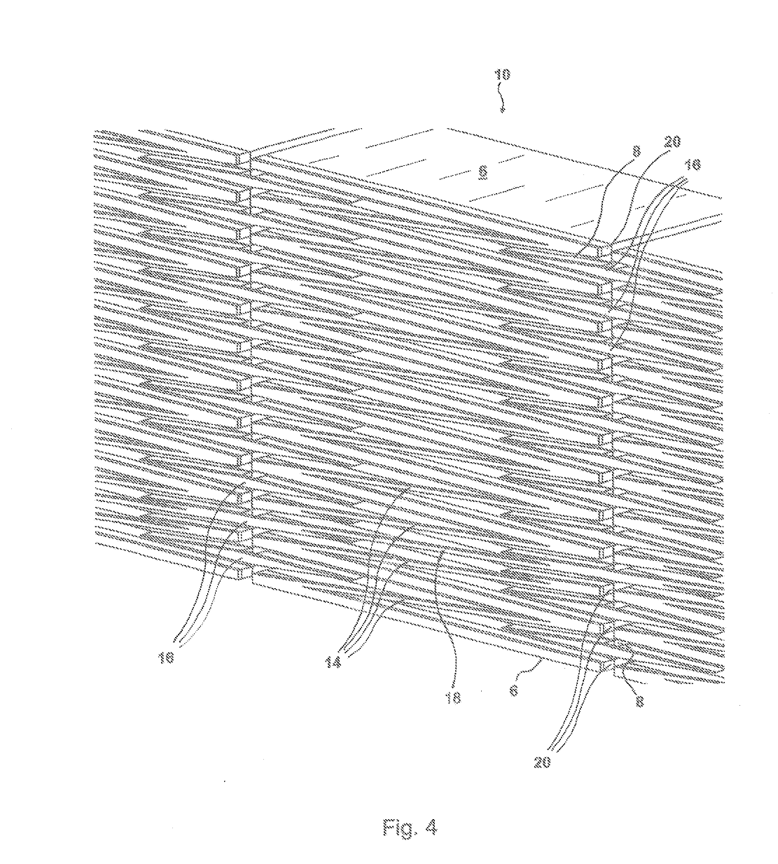

As can be seen from FIGS. 3 and 4, and also FIG. 6, the control electrodes 6 and the reference electrodes 8 are in each case designed with a plate-like shape, i.e., they have a greater extent in the radial direction R in relation to the longitudinal axis X than in the direction of the longitudinal axis X. This brings about a rigid design of the electrodes 6 and 8, particularly in the radial direction. As a result of this, the reference electrodes 8 and the control electrodes 6 at the same time bring about a strengthening in the radial direction of the wall made of electroactive polymer. However, the elastic webs 14 and 18 allow movement in the axial direction X of the reference electrodes 8 and the control electrodes 6 toward one another if the electroactive polymer situated therebetween is deformed in this direction. This implements the flexibility of the shaft.

The implementation of the structure consisting of control electrodes 6 and reference electrodes 8, and also the webs 14, 16 and 18 is shown in FIGS. 3 and 4. FIGS. 3 and 4 show that the electrode structure is preferably at first designed as a planar structure, i.e., it is not bent to a tube. The electrode structure thus formed can then be molded into the electroactive polymer and, together with the latter, can be bent into the tube-like shape shown in FIG. 6 (FIG. 6 does not illustrate the electroactive polymer). Subsequently, this structure can then once again be encapsulated on the outside by an elastic polymer in order to fix the shape thus formed.

FIG. 6 shows that a multiplicity of actuator fields 10 can be arranged uniformly distributed over the circumference of the tube around the longitudinal axis X, such that the shaft can be deflected in different radial directions with very precise control. At the same time, the reference electrodes 8 and control electrodes 6 of the individual actuator fields 10 almost adjoin one another directly, such that electrodes are arranged in all circumferential regions, and so the wall is stabilized in the radial direction in all circumferential regions. FIG. 6 shows that the electrode arrangement according to FIG. 3 has been bent such that the elastic webs 14 and 18 are situated on the internal circumference of the electrode arrangement. This keeps the deformation paths for the elastic webs short during the deformation.

FIGS. 3 and 4 show how the connection webs are attached to the plate-shaped control electrodes 6 and reference electrodes 8. At the end edges, which later face the inner circumference relative to the longitudinal axis X, the webs are attached via electrically conducting spacers 20. The effect of the spacers 20 is that the elastic webs 14 can laterally pass by the reference electrodes 8 situated therebetween without touching the latter. It is correspondingly ensured that the elastic webs 18 can laterally pass the control electrodes 6 situated therebetween without touching the electrodes.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. An endoscopic instrument (2) comprising a shaft (3) designed in a flexible manner along its longitudinal axis (X) in at least one section (4) of the shaft, the flexible section (4) of the shaft (3) having a tube-shaped wall made of an electroactive polymer with a plurality of control electrodes (6) and reference electrodes (8) respectively embedded therein, the control electrodes (6) and the reference electrodes (8) being arranged alternately as seen in the axial direction (X) and being separated from one another, and the control electrodes (6) and reference electrodes (8) alternating in the axial direction (X) respectively having a rigid design and being respectively interconnected in an electrically conducting manner in the axial direction (X) by elastic webs (14, 18) arranged on the outer or inner circumference of the flexible section of the shaft.

2. The endoscopic instrument according to claim 1, wherein the control electrodes (6) and/or the reference electrodes (8) have a plate-shaped design and extend transversely relative to the longitudinal axis (X).

3. The endoscopic instrument according to claim 1, wherein the flexible section (4) of the shaft (3), as seen in a circumferential direction, has at least two actuator fields (10) activatable separately, in which fields, as seen in the axial direction (X), alternating control electrodes (6) and reference electrodes (8) are respectively arranged.

4. The endoscopic instrument according to claim 3, wherein the reference electrodes (8) of the at least two actuator fields (10) are interconnected in an electrically conducting manner.

5. The endoscopic instrument according to claim 4, wherein the reference electrodes (8) of the at least two actuator fields (10) respectively situated in one diameter plane are interconnected in the circumferential direction in an electrically conducting manner.

6. The endoscopic instrument according to claim 4, wherein the reference electrodes (8) of the at least two actuator fields (10) respectively situated in one diameter plane are interconnected in the circumferential direction in a mechanical manner.

7. The endoscopic instrument according to claim 4, wherein the reference electrodes (8) of the at least two actuator fields (10) have a common electrical connection (18) in the axial direction (X).

8. The endoscopic instrument according to claim 7, wherein first and second reference electrodes (8) adjacent to one another in the axial direction (X) are interconnected by an elastic web (18), which, as seen in the circumferential direction, is connected to a first end of the first reference electrode (8) and an opposite second end of the second reference electrode (8).

9. The endoscopic instrument according to claim 7, wherein the common axial electrical connection (18) of the reference electrodes (8) is arranged alternately distributed in the at least two actuator fields (10).

10. The endoscopic instrument according to claim 9, wherein the common axial electrical connection of the reference electrodes (8) is designed as a web (18) running in a wall of the at least one section embedded in a helical manner.

11. The endoscopic instrument according to claim 3, wherein two control electrodes (6) respectively adjacent to one another in the at least two actuator fields (10) are interconnected by an elastic web (14), which, as seen in the circumferential direction, is connected to a first end of a first control electrode (6) and an opposite second end of a second adjacent control electrode (6).

12. The endoscopic instrument according to claim 1, wherein the elastic webs (18), which interconnect the reference electrodes (8), and/or the elastic webs (14), which interconnect the control electrodes (6), are arranged on a circumferential side of the control electrodes (6) and reference electrodes (8) situated radially on the inside.

13. A method for producing an endoscopic instrument according to claim 1, the method comprising:
first arranging the control electrodes (6) and reference electrodes (8) and the elastic webs (14, 18) connecting these as a structure in a flat plane, wherein control electrodes (6) and reference electrodes (8) alternate in a direction of extent of the plane;
subsequently molding the structure into an electroactive polymer; and
then bending the molded structure to form a tube-shaped wall.

14. The method according to claim 13, further comprising encapsulating the tube-shaped wall by an elastic polymer on its outer side after the bending.
